(19) United States
(12) Patent Application Publication
(10) Pub. No.: US 2026/0266752 A1
(43) Pub. Date: Sep. 10, 2026

(54) MEASUREMENT DEVICE FOR DETERMINING DIELECTRIC CONSTANT AND CONDUCTIVITY

(71) Applicant: Endress+Hauser SE+Co. KG, Maulburg (DE)

(72) Inventors: Thomas Blödt, Steinen (DE); Peter Klöfer, Steinen (DE); Sylwester Szymanski, Schopfheim (DE)

(21) Appl. No.: 19/164,064

(22) PCT Filed: Feb. 16, 2024
(86) PCT No.: PCT/EP2024/053995

§ 371 Date: Sep. 10, 2025

(30) Foreign Application Priority Data
Mar. 14, 2023 (DE) 10 2023 106 371.5

(51) Int. Cl. G01N 27/22

(52) U.S. Cl. CPC G01N 27/221; G01N 27/226; G01N 27/228

(57) ABSTRACT

A measurement device, for determining both the dielectric constant and the electrical conductivity of a fill substance includes two electrodes that may contact the fill substance and a high frequency measuring unit that couples a high frequency signal into the first electrode and receives the high frequency signal at the second electrode after interaction of the high frequency signal with the fill substance. The dielectric constant of the fill substance is determined based on the received high frequency signal. A conductivity measuring unit couples a supplemental measuring signal into one of the electrodes and couples the supplemental measuring signal out of the other electrode after interaction of the supplemental measuring signal with the fill substance. The conductivity of the fill substance is determined based on the out-coupled measuring signal. The measurement device includes a signal splitting unit to separate the high frequency and supplemental signals from one another.

The invention relates to a high frequency based measurement device for determining dielectric constant and conductivity of a fill substance.

In automation technology, especially for process automation, field devices are often applied, which for serve registering diverse measured variables. The measured variables can be, for example, fill level, flow, pressure, temperature, pH value, redox potential, conductivity or dielectric constant of a fill substance in a process plant. For registering the corresponding measured values, the field devices comprise suitable sensors based on suitable measuring methods. A large number of different field device types are manufactured and sold by the Endress+Hauser group of firms.

The determination of dielectric constant (also known as “dielectrical number” or “relative permittivity”) of diverse media is of great interest both in the case of solids, as well as also in the case of liquid and gaseous fill substances, such as, for example, fuels, waste waters, gases, gas phases and chemicals, since such can be a reliable indicator for impurities, moisture content, substance concentration, or substance composition. The dielectric constant of a fill substance can be determined using high frequency, for example, by measuring amplitude, phase shift or signal travel time of high frequency signals in the case of passage through the fill substance. In such case, a high frequency signal of defined frequency or defined frequency band, is coupled into the fill substance. After passage through the fill substance, the high frequency signal is evaluated relative to its amplitude, phase difference or signal travel time compared with the initial high frequency signal. The terminology, “high frequency signal”, means, in the context of the invention, signals with frequencies between 10 MHz and 150 GHz. A phase based dielectric constant measuring device is described, for example, in WO 2022033831 A1.

Especially in the pharma- and food industries, it is helpful for process control, when, besides the dielectric constant, also conductivity of the process-participating fill substances is determined and/or monitored. It is, however, more disadvantageous from a hygienic point of view that more measurement devices need to be brought in contact with the fill substance. For each additional flange connection on the process container required for a corresponding measurement device, such represents from a hygienic point of view a potential germ-source and needs correspondingly to be complexly designed.

An object of the invention, therefore, is to be able to determine dielectric constant and conductivity of fill substances with as few measurement devices as possible.

The invention achieves the object by a measurement device for determining dielectric constant and electrical conductivity of a fill substance, comprising:
a first electrode, which can be brought into galvanic contact with the fill substance,
a second electrode, which can be brought into galvanic contact with the fill substance,
a high frequency measuring unit, which is designed
   to couple via a high frequency output an electrical high frequency signal into the first electrode,
   to receive via a high frequency input the high frequency signal from the second electrode after interaction of the high frequency signal with the fill substance, and
   to determine at least based on the received high frequency signal the dielectric constant of the fill substance,
a conductivity measuring unit, which is designed,
   to couple via a signal output a measuring signal into one of the electrodes,
   to couple via a signal input the measuring signal out of the other electrode after interaction of the measuring signal with the fill substance, and
   to determine the conductivity of the fill substance based on the out-coupled measuring signal, and
a signal decoupling unit, which is designed to separate the signals of the high frequency measuring unit and the conductivity measuring unit from one another.

The invention is, thus, based on the idea of using the electrodes, by means of which the dielectric constant is determined with high frequency, also for determining conductivity. Thus, no separate electrodes and no additional measurement device are needed, in order to be able to determine, besides the dielectric constant, the conductivity of the fill substance. In this way, the fill substance is accessed less from the outside, whereby, in turn, the hygienic boundary conditions in the container are improved.

The high frequency measuring unit can ascertain the dielectric constant either in the form of the imaginary part and the real part, or as a magnitude. Depending on design of the high frequency measuring unit, the determining of the dielectric constant can occur based on amplitude, signal-, or signal group travel time, signal quality and/or pulse-, or frequency response of the received high frequency signal. In such case, the frequency of the high frequency signal is to be chosen, in principle, as a function of the dielectric constant measuring range. In the case of a measuring range of the dielectric constant between 60 and 90, thus, in the case of greatly moisture containing media, the high frequency measuring unit is to be designed to produce the electrical high frequency signal for this measuring range with a frequency between 0.1 GHZ and 30 GHZ, especially between 2 GHZ and 8 GHz.

As regards the conductivity determination in the context of the invention, in principle, any measuring principle can be used, which enables ascertaining the conductivity using two electrodes. Accordingly for example, the capacitive, conductive, or transmittive measuring principle can be implemented in the conductivity measuring unit, since all these principles operate based on the application of two electrodes. For example, a process automation measurement device based on the capacitively conductive measuring principle is described in WO 2019141464 A1.

Also the implementing of the signal decoupling unit is not fixedly prescribed within the scope of the invention, as long as the signals of the high frequency measuring unit and the conductivity measuring unit are sufficiently separated from one another. In such case, the design of the signal decoupling unit is governed in part by how the measuring units are implemented. Depending on this, the signal decoupling unit can comprise, for example, a first diodes arrangement connected in the signal direction before-, respectively following, the high frequency output and/or the high frequency input. Additionally or alternatively, the signal decoupling unit can comprise a second diodes arrangement connected in the signal direction before-, respectively following, the signal output and/or the signal input. In such case, the terminology “diode” includes also correspondingly connected transistors as well as corresponding electronic components with equivalent function.

Alternatively or supplementally to diodes, the signal decoupling unit can, moreover, be equipped with a first signal splitter connected in front of the first electrode, and/or a second signal splitter connected in front of the second electrode. In such case, for example, a circulator, a duplexer and/or a diplexer can function as a signal splitter.

Another option is, moreover, that the signal decoupling unit for decoupling of the measuring units comprises a first frequency filter connected in front of the high frequency measuring unit and/or a second frequency filter connected in front of the conductivity measuring unit.

In this connection, it is an option to establish the signal decoupling unit not only based on frequency filters, signal splitters or diodes arrangements, but, instead, mixed. I.e., the signal decoupling unit includes, in such case, besides one or more frequency filters additionally one or more signal splitters, or diodes. Also the mixed design of the signal decoupling unit can be done based on signal splitter(s) and diodes arrangement(s).

Independently of which electrical components the signal decoupling unit comprises for signal separation, there is, depending on design of the measuring units, another design variable in that either of the outputs of the two measuring units are connected via the signal decoupling unit to the first electrode, wherein, in such case, the inputs of the two measuring units are connected to the second electrode (again, in each case, via the signal decoupling unit). Or, the signal outputs of the conductivity measuring unit and the high frequency unit are connected via the signal decoupling unit, in each case, to the second electrode, while the high frequency input and the conductivity signal input are connected to the first electrode, in each case, via the signal decoupling unit.

The construction of the signal decoupling unit based on frequency filters, signal splitters, and/or diodes arrangements rests on implementing the signal separation between the measuring units and the electrodes. An alternative or supplemental design for this is to operate the measuring units delayed relative to one another. I.e., the signal decoupling unit is, in such case, designed to control the high frequency measuring unit and the conductivity measuring unit, for example, by means of a control signal, in such a manner that, in each case, one of the units is active and the other inactive.

In general, the terminology “unit” in the context of the invention means, in principle, any electronic circuit suitably designed for the intended application. It can, thus, depending on requirement, be an analog circuit for producing and/or processing corresponding analog signals. It can even be a digital circuit, such as an FPGA or a storage medium in cooperation with a program. In such case, the program is designed to perform the corresponding method steps, or to apply the needed computer operations of the particular unit. In this context, different electronic units of the measurement device can, within the scope of the invention, potentially also access a shared physical memory, or be operated by means of the same physical, digital circuit.

The invention will now be explained in greater detail based on the appended drawing, the figures of which show as follows:

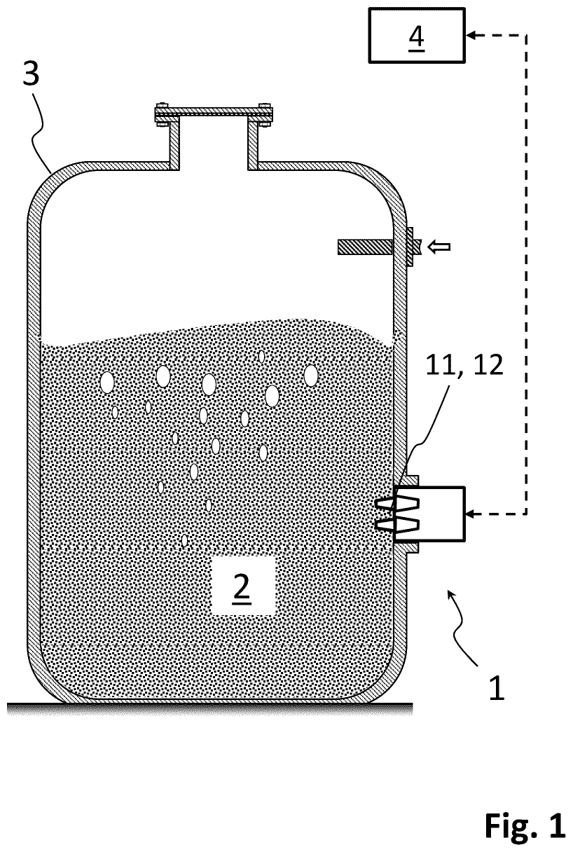

FIG. 1 a measurement device of the invention for conductivity- and dielectric constant measurement of a fill substance in a container,

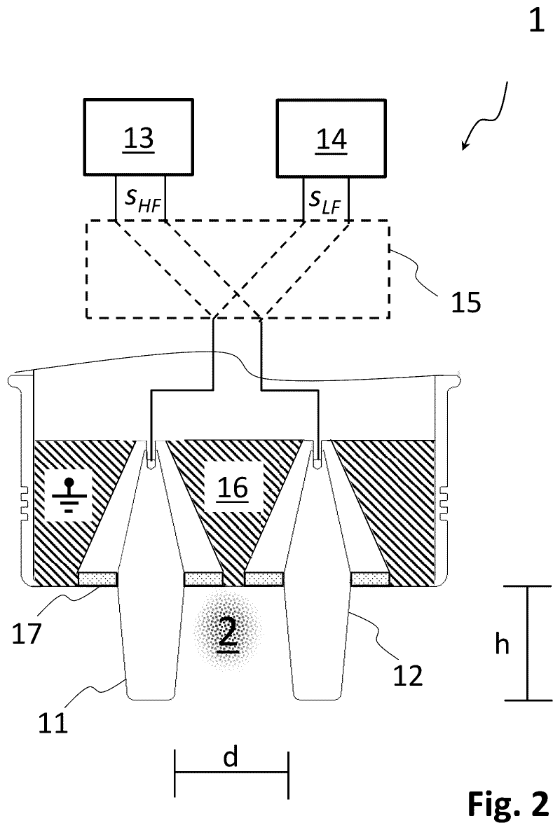

FIG. 2 a detail view of the measurement device with signal decoupling unit,

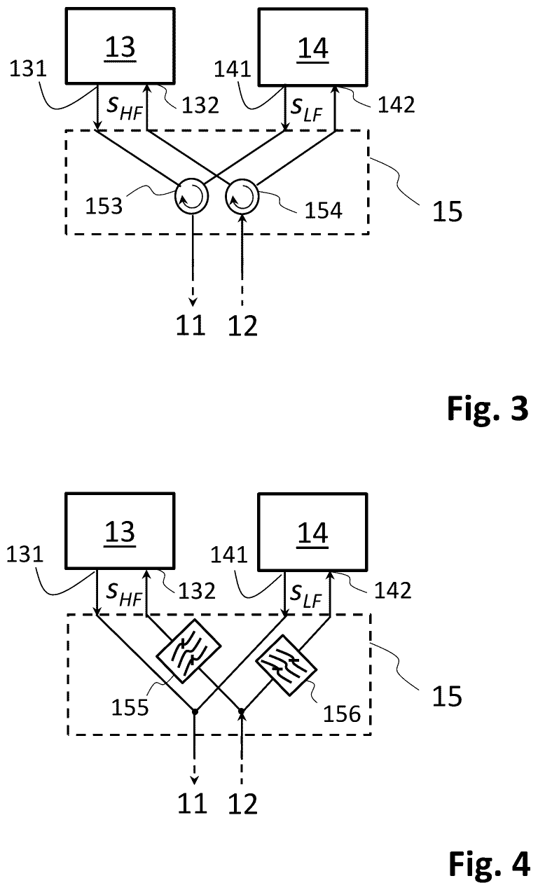

FIG. 3 a first embodiment of the signal decoupling unit,

FIG. 4 a second embodiment of the signal decoupling unit,

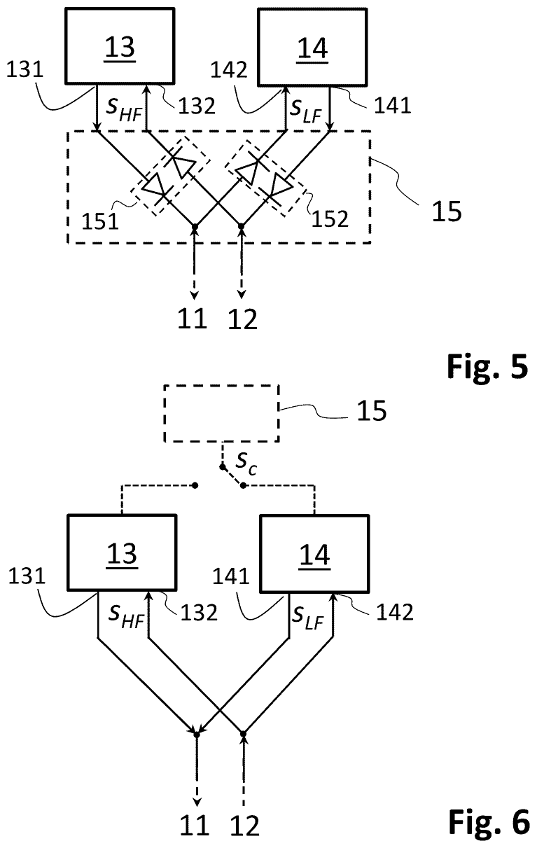

FIG. 5 a third embodiment of the signal decoupling unit, and

FIG. 6 a fourth embodiment of the signal decoupling unit.

For understanding the invention, FIG. 1 shows a container 3 applied, for example, in a pharmaceutical production process. Container 3 is filled with a fill substance 2, whose dielectric constant and conductivity are to be determined. For this, a measurement device 1 of the invention is secured via a lateral, external port of the container 3, via, for example, a flange of size DN50, in such a manner, that two electrodes 11, 12 protrude into the container interior and penetrate into the fill substance 2. Optionally, the measurement device 1 can be connected to a superordinated unit 4, such as, for example, a process control system. Provided as interface can be, for instance, a “PROFIBUS”, “HART”, “WirelessHART” or “Ethernet” connection. Measured values can be transmitted in this way. Also, other information concerning general operating state of the measurement device 1 can be communicated.

In the illustrated example of an embodiment, dielectric constant and conductivity of the fill substance 2 are ascertained by the measurement device 1 of the invention, in each case, based on transmittive measurement. For the dielectric constant, high frequency signals s_{HF} are transmitted via one of the electrodes 11, which functions as a first electrode 11. The second electrode 12 serves for receiving the high frequency signals s_{HF} after their passage through the fill substance 2. In such case, the measurement path d, which the high frequency signals s_{HF} travel, is determined by the distance d between the electrodes 11, 12. As can be seen from FIG. 2, the electrodes 11, 12 for transmitting and receiving extend to a defined depth h into the container interior and into the fill substance 2. In such case, the high frequency signals s_{HF} are produced in a correspondingly designed, high frequency measuring unit 13 of the measurement device 1 and fed via a high frequency output 131 to the first electrode 11. The second electrode 12 is connected with the high frequency measuring unit 13 via a high frequency input 132, in order correspondingly to evaluate the high frequency signals s_{HF} upon their receipt,

In the context of the invention, it does not matter whether the electrodes 11, 12 are manufactured completely of a conductive material, such as, for example, turned stainless steel, or whether the electrodes 11, 12 have only an electrically conductive surface coating. A metallizing of the electrode surfaces can be deposited, for example, by means of plasma coating, such as PECVD (“plasma enhanced vapor deposition”).

Based on the amplitude of the received high frequency signal s_{HF}, the high frequency measuring unit 13 can determine the real part of the dielectric constant. Based on the signal travel time and the phase difference of the received high frequency signal s_{HF}, the imaginary part of the dielectric constant can be determined. In such case, implemented as measuring principle for determining the signal travel time can be, for example, the pulse travel time method or the FMCW method, analogously to radar based distance measurement. The high frequency measuring unit 13 is constructed according to the applied measuring principle.

Especially in the foods- and pharmaceuticals industry, the fill substance 2 can be significantly water containing liquids, such as drinks or vaccines. Accordingly, the dielectric constant range to be registered in these cases lies between 60 and 90. Corresponding to this region, the high frequency measuring unit 13 is preferably designed to produce the high frequency signals s_{HF} with a frequency f between 2 GHz and 8 GHZ. Naturally in the case of other dielectric constant measurement ranges, other frequencies can be implemented.

In order that especially in the dielectric constant range between 60 and 90 a high resolution can be achieved, preferably the near field of the high frequency signal s_{HF} is coupled out from the first electrode 11. Advantageous here is the low attenuation in media 2 with high dielectric constants and the high measuring sensitivity associated therewith. Moreover, disturbing effects of the far field are avoided, such as, for example, undesired reflections of the inner surface of the container 2, which can corrupt the measurement. For this, the first electrode 11 is constructed with a depth h for predominant radiation in the near field, which according to

$$c = \frac{f * λ}{\sqrt{DK}}$$

is essentially less than one fourth the wavelength λ of the high frequency signal s_{HF}, thus, for example, an eighth of the wavelength λ. In such case, c is the propagation velocity of the high frequency signal s_{HF} in the fill substance 2; DK is the dielectric constant of the fill substance 2. By this selection of the electrodes depth h, it is also assured that the measurement device 1 can be designed with compact dimensions. In this way, the measurement device can be installed at small container openings. The depth h of the electrodes 11, 12 here is with reference to a measurement device planar wall 16, which faces toward fill substance 2 and the container interior and which functions in the illustrated example of an embodiment at the same time as signal ground for the high frequency signal s_{HF}. Accordingly, wall 16 can be made of a stainless steel, for example. A minimum depth h of the electrodes 12, 13 from the wall 16 is not fixedly predetermined. In principle, it is even an option that the electrodes 11, 12 not protrude from the wall 16 into the container interior. An advantage of a depth h greater than zero is, however, the higher sensitivity of the dielectric constant measurement.

The distance d between the first electrode 11 and the second electrode 12 amounts preferably to maximum of a fourth and minimum of an eighth of the wavelength λ corresponding to the frequency f of the high frequency signal s_{HF} in the above formula. In this way, the effect is utilized, according to which the first electrode 11 transmits the high frequency signal s_{HF} into the distance λ/8<d<λ/4 with the highest field density. The distance “d” is measured, in such case, at those two points on the surfaces of the electrodes 11, 12 having the smallest distance from one another.

As a result of this positioning of the second electrode 12 relative to the first electrode 11, the sensitivity of the dielectric constant measurement is maximized. This effect is supported by providing the two electrodes 11, 12 with the same geometry, and the same depth h and/or a conical tapering narrower with increasing depth h, such as shown in FIG. 2. In the case of the embodiment shown in FIG. 2, the two electrodes 11, 12 have, additionally, ends that are rounded off. This is advantageous especially in the case of hygienically sensitive applications, in the case of which fill substance deposits are to be prevented. Such is also advantageous for applications, in the case of which the fill substance 2 is not stationary in the container 3, but, instead, for example, flows through a pipeline section, in order to suppress vortex formation in the pipeline section, when the measurement device is arranged therein. The cross sectional shape of the electrodes 11, 12 is not fixedly predetermined within the scope of the invention. In principle, the cross section can have, for example, a round, elliptical or rectangular shape.

Electrodes 11, 12 are electrically isolated, in each case, by an electrical insulation 17, which separates the electrode 11, 12 from the wall 16. In such case, an electrical insulation 17 can be implemented, for example, as an injection molded part. The material can be, for example, PP, PTFE, PEEK or a ceramic can be used, such as aluminum oxide. In the case of the example of an embodiment shown in FIG. 2, the two insulations 17 are designed such that they flushly close wall 16 against the fill substance 2. Moreover evident from FIG. 2 is that the insulations 17 of the electrodes 11, 12 are, in each case, separated by the wall 16 functioning as signal ground. In this way, the supplementally advantageous effect is achieved that the high frequency signal s_{HF} must travel through the fill substance 2 completely, without, at least partially, being able to couple directly from the first electrode 11 into the second electrode 12. Thus, the sensitivity of the dielectric constant measurement is further increased.

According to the invention, not only the dielectric constant of the fill substance 2 is determined by means of the electrodes 11, 12, but, supplementally, its conductivity. In this way, it becomes unnecessary to mount a separate conductivity measurement device on the container 3, whereby better hygienic conditions are created. As shown in FIG. 2, measurement device 1 includes for this, besides the high frequency unit 13, additionally, a conductivity measuring unit 14, which is connected with one of the electrodes 11, 12 via a signal output 141. A signal input 142 of the conductivity measuring unit 14 is connected with the other electrode 11, 12. Analogously to the high frequency unit 13, the conductivity measuring unit 14 couples via the signal output 141 a measuring signal s_{LF} into the corresponding electrode 11, 12 and receives such with the other electrode 11, 12 after interaction with the fill substance 2. In this way, the conductivity measuring unit 14 can ascertain the electrical conductivity of the fill substance 2 based on the received measuring signal s_{LF} especially according to the capacitively-conductive measuring principle.

In order to be able to determine both the conductivity as well as also the dielectric constant via the two electrodes 1, 12, the measurement device 1 of the invention includes a signal decoupling unit 15, which separates the high frequency signal s_{HF} of the high frequency measuring unit 13 from the measuring signal s_{LF} of the conductivity measuring unit 14. As illustrated based on FIGS. 3 to 6, a number of embodiments are available, in principle, for this:

The embodiment of the signal decoupling unit 15 shown in FIG. 3 includes two circulators 153, 154 placed in front of the electrodes 11, 12. Any other forms of signal splitters can be used instead of the circulators 153, 154. In the case of the embodiment shown in FIG. 3, the signal outputs 131, 141 of the two measuring units 13, 14 are electrically connected via the first circulator 153 with the first electrode 11, while the signal inputs 132, 142 are electrically connected via the second circulator 154 with the second electrode 12. Instead of this setup, it is naturally also an option that the signal output 131 of the high frequency measuring unit 13 and the signal input 142 of the conductivity measuring unit 14 are connected via the first circulator 153 with the first electrode 11, while the signal input 132 of the high frequency measuring unit 13 and the signal output 141 of the conductivity measuring unit 14 are connected via the second circulator 154 with the second electrode 12.

In the case of the embodiment shown in FIG. 4, the signal decoupling unit 15 is based on frequency filters 155, 156 placed in front of the signal inputs 132, 142. In such case, the first frequency filter 155 is placed in front of the signal input 132 of the high frequency measuring unit 13 and designed as a bandpass filter in such a manner that only the frequency of the high frequency signal s_{HF} is let through. The second bandpass filter 156 is placed in front of the signal input 142 of the conductivity measuring unit 14 and is transmissive for the frequency of its measuring signal s_{LF}. Instead of constructing the frequency filters 155, 156 as band pass filters, it is, alternatively, also an option to construct the first frequency filter 155 as a highpass for the high frequency signal s_{HF}, while the second frequency filter 156 forms a lowpass filter for the measuring signal s_{LF}.

Also in the case of the embodiment shown in FIG. 4, the signal outputs 131, 141 of the measuring units 13, 14 are connected with the first electrode 11, while the signal inputs 132, 142 are connected via the frequency filters 155, 156 with the second electrode 12. Analogously to FIG. 3, it is also an option as regards the form of embodiment shown in FIG. 4 that the signal output 131 of the high frequency measuring unit 13 and the signal input 142 of the conductivity measuring unit 14 are connected with the first electrode 11, while the signal input 132 of the high frequency measuring unit 13 and the signal output 141 of the conductivity measuring unit 14 are connected with the second electrode 12. Also in this case, the frequency filters 155, 156 are connected in front of the signal inputs 132, 142.

Instead of connecting the frequency filters 155, 156 in front of the signal inputs 132, 142 of the measuring units 13, 14, it is, moreover, an option to connect the frequency filters 155, 156 downstream of the signal outputs 131, 141. The resulting bandwidth limitations lead to a lessening of noise.

The embodiment of the signal decoupling unit 15 shown in FIG. 5 is based on two diodes arrangements 151, 152 arranged between the measuring units 13, 14 and the electrodes 11, 12. In such case, the first diodes arrangement 151 is associated with the high frequency measuring unit 13 and includes two diodes, of which the first diode is connected, in the signal direction, following the high frequency output 131 and in front of the first electrode 11. The second diode of the first diodes arrangement 151 is placed in the signal direction in front of the high frequency input 132 and following the second electrode 12. The second diodes arrangement 152 is associated with the conductivity measuring unit 14. Corresponding to the first diodes arrangement 151, a third diode of the second diodes arrangement 152 is connected downstream from the signal output 141 of the conductivity measuring unit 14. A fourth diode of the second diodes arrangement 152 is connected before the signal input 142 of the conductivity measuring unit 14.

Of course, the embodiments of the signal decoupling unit 15 shown in FIGS. 3 to 5 are implementable within the scope of the invention not just separately. Thus, an illustrated form of embodiment can for a better signal separation include, for example, supplementally elements of other forms of embodiment: For example, the variant of the signal decoupling unit 15 shown in FIG. 3 can be supplemented between the circulators 153, 154 and the measuring units 13, 14 with the frequency filters 155, 156.

In contrast with the embodiments shown in FIGS. 3 to 5, the signal decoupling unit 15 of the form of embodiment of FIG. 6 is not arranged between the measuring units 13, 14 and the electrodes 11, 12.

Rather, the signal decoupling unit 15 in the case of this form of embodiment controls the measuring units 13, 14 in such a manner that they measure shifted in time from one another: This means that one of the measuring units 13, 14 is active for a defined period and measures its measured value, while the other unit 13, 14 is inactive within this period and does not measure. After end of this period, the other measuring unit 13, 14 is activated for a period following thereon and the previously active measuring unit is deactivated. Correspondingly, the measuring units 13, 14 are, depending on their design, controlled by the signal decoupling unit 15. For example, they can be switched on and off via a control signal s_{c} or a periodically switchable switch, such as shown in FIG. 6. Also this form of embodiment of the signal decoupling unit 15 can for the purpose of further improved signal separation be combined with one of the forms of embodiment described based on FIGS. 3 to 5.

### LIST OF REFERENCE CHARACTERS

1 measurement device
2 fill substance
3 container
4 superordinated unit
11 first electrode
12 second electrode
13 high frequency measuring unit
14 conductivity measuring unit
15 signal decoupling unit
16 wall
17 electrical insulation
131 high frequency output
132 high frequency input
141 signal output
142 signal input
151 first diodes arrangement
152 second diodes arrangement
153 first signal splitter
154 second signal splitter
155 first frequency filter
156 second frequency filter
d distance between the electrodes
f frequency of the high frequency signal
h depth of the electrodes
s_{HF} high frequency signal
s_{LF} measuring signal
s_{c} control signal
λ wavelength of the high frequency signal

1-8. (canceled)

9. A measurement device for a determining dielectric constant and an electrical conductivity of a fill substance, comprising:
a first electrode that can be brought into galvanic contact with the fill substance;
a second electrode that can be brought into galvanic contact with the fill substance;
a high frequency measuring unit that is designed to couple via a high frequency output an electrical high frequency signal into the first electrode, to receive via a high frequency input the high frequency signal from the second electrode after interaction of the high frequency signal with the fill substance, and to determine at least based on the received high frequency signal the dielectric constant of the fill substance;
a conductivity measuring unit that is designed to couple via a signal output a measuring signal into one of the electrodes, to couple via a signal input the measuring signal out of the other electrode after interaction of the measuring signal with the fill substance, and to determine the conductivity of the fill substance based on the out-coupled measuring signal; and
a signal decoupling unit that is designed to separate the signals of the high frequency measuring unit and the conductivity measuring unit from one another.

10. The measuring device as claimed in claim 9, wherein the high frequency measuring unit is designed to determine an imaginary part, a real part, or a magnitude of the dielectric constant based on an amplitude, a signal or signal group travel time, a signal quality, and/or a pulse or frequency response of the received high frequency signal.

11. The measuring device as claimed in claim 9, wherein the high frequency measuring unit is designed to produce the electrical high frequency signal with a frequency between 0.1 GHz and 30 GHz.

12. The measuring device as claimed in claim 9, wherein the conductivity measuring unit is designed to determine the conductivity of the fill substance via a capacitive, conductive, and/or transmissive measuring principle.

13. The measuring device as claimed in claim 9, wherein the signal decoupling unit includes a first diodes arrangement connected in the signal direction before the high frequency output and/or following the high frequency input, and/or a second diodes arrangement connected in the signal direction before the signal output and/or following the signal input.

14. The measuring device as claimed in claim 9, wherein the signal decoupling unit includes a first signal splitter connected in front of the first electrode, and/or a second signal splitter connected in front of the second electrode.

15. The measuring device as claimed in claim 9, wherein the signal decoupling unit includes a first frequency filter connected in front of the high frequency measuring unit and/or a second frequency filter connected in front of the conductivity measuring unit.

16. The measuring device as claimed in claim 9, wherein the signal decoupling unit is designed to control the high frequency measuring unit and the conductivity measuring unit by a control signal in such a manner that, in each case, one of the measuring units is active and the other measuring unit inactive.
